(19) United States
(12) Patent Application Publication
(10) Pub. No.: US 2026/0076641 A1
(43) Pub. Date: Mar. 19, 2026

(54) DEVICE AND METHOD FOR CONTINUOUS, NON-INVASIVE BLOOD PRESSURE MONITORING

(71) Applicant: AGOATECH, LLC, Kirkland, WA (US)

(72) Inventors: Eric Afonso, Kirkland, WA (US); Luis Afonso, Dearborn, MI (US)

(21) Appl. No.: 19/331,225

(22) Filed: Sep. 17, 2025

Related U.S. Application Data
(60) Provisional application No. 63/695,512, filed on Sep. 17, 2024.

(51) Int. Cl. A61B 8/04; A61B 8/00; A61B 8/06; B06B 1/02; B06B 1/06

(52) U.S. Cl. CPC A61B 8/04; A61B 8/06; A61B 8/4236; A61B 8/488; A61B 8/565; A61B 8/58; B06B 1/0292; B06B 1/06

(57) ABSTRACT

A continuous, non-invasive, blood pressure monitoring system for determining a blood pressure measurement for a wearer is provided. The system has accuracy close to that of an invasive catheter transducer system for continuous blood pressure measurement. The system includes an ultrasonic sensor patch, a processor, and a memory. The patch includes an ultrasonic transducer located externally on a body part of the wearer and in a vicinity of a vessel that is configured to determine a diameter of the vessel. The patch also includes a power component. Further, the memory includes instructions that, when executed by the processor, cause the system to calculate a cuffless blood pressure measurement for the wearer based on the diameter of the vessel as determined by the transducer and transmit the cuffless blood pressure measurement to a host computer via a network. A method for continuous, non-invasive, blood pressure measurement is also provided.

### RELATED APPLICATIONS

The present application claims priority to U.S. Provisional Patent Application No. 63/695,512, filed on Sep. 17, 2024, the entire contents of which are incorporated herein by reference.

### TECHNICAL FIELD

The subject matter disclosed here in is generally directed to a device for blood pressure measurement. In particular, the device can provide for continuous, non-invasive blood pressure measurement and/or monitoring without the use of a blood pressure cuff.

### BACKGROUND OF THE INVENTION

Hypertension, or high blood pressure, is a condition that puts one at risk for heart disease and stroke, which are two of the leading causes of death in the United States. It is estimated that one in every two American adults is hypertensive, and more than half of hypertensive patients have uncontrolled BP. The cost associated with managing and treating such patients exceeds $125 billion dollars. Despite this major health concern, developments in BP measurement and monitoring have lagged behind other advances in personal health monitoring.

Currently, accurately measuring a person's blood pressure (BP) is a key vital sign in order to treat hypertension. The generally accepted standard practice for measuring BP non-invasively includes two techniques: auscultatory and oscillometric methods. BP devices that utilize either method, however, have been proven to be inaccurate, especially for hypertensive cases. Both techniques underestimate systolic pressure and overestimate diastolic pressure in a large percentage of patients. Meanwhile, invasive monitoring, while accurate, requires a hospital setting and cannot be used to monitor a patient's BP while the patient is at home.

Therefore, physicians have identified a need for an improved BP device with increased reliability and patient compliance for at home BP measurement. Additionally, nighttime blood pressure variability (BPV) is of increasing significance in patient care, and physicians lack the tools to accurately monitor it. In particular, physicians typically prescribe home ambulatory blood pressure monitoring devices (ABPM) to monitor the patient in a home environment free of doctor's office anxiety. ABPM devices provide the ability to track blood pressure data throughout the day and night. However, the best available technologies for ABPM are oscillometric devices that are known to be inaccurate for hypertensive patients. The intermittent data (4-5 spot checks per hour) collected by these devices does not provide enough value to effectively treat the patient and the device itself is cumbersome to the patient. Meanwhile, a continuous non-invasive BP (CNIBP) would be more advantageous than spot checks in the amount of data that can be collected and analyzed for patient electronic health records (EHR) and treatment.

As such, a need currently exists for a non-invasive device for measuring and/or monitoring blood pressure continuously. Such a device that does not require the use of a bulky, cumbersome blood pressure cuff would be useful and increase blood pressure measurement accuracy.

### SUMMARY OF THE INVENTION

Aspects and advantages of embodiments of the present disclosure will be set forth in part in the following description, or can be learned from the description, or can be learned through practice of the embodiments.

In one embodiment, a continuous, non-invasive, blood pressure monitoring system for determining a blood pressure measurement for a wearer is contemplated by the present disclosure. The system includes an ultrasonic sensor patch, at least one processor, and a first memory. The ultrasonic sensor patch includes at least one ultrasonic transducer located externally on a body part of the wearer and in a vicinity of a vessel, the at least one ultrasonic transducer configured to determine a diameter of the vessel, as well as a power component. The first memory includes instructions that, when executed by the at least one processor, cause the continuous, non-invasive blood pressure monitoring system to: calculate a cuffless blood pressure measurement for the wearer based on the diameter of the vessel as determined by the at least one ultrasonic transducer; and transmit the cuffless blood pressure measurement to a host computer via a network.

In another aspect, the system can include a calibration system. The calibration system can include an inflatable cuff; at least one controller; a compressor in communication with the inflatable cuff and configured to inflate or deflate the cuff; a pressure transducer configured to measure pressure levels to which the inflatable cuff is inflated or deflated in real time; an ultrasonic Doppler sensor located externally on the body part of the wearer in the vicinity of the vessel and positioned proximal to the inflatable cuff, the ultrasonic Doppler sensor configured to determine a velocity of blood flow within the vessel in response to the inflation or deflation of the inflatable cuff; and a second memory including instructions that, when executed by the at least one controller, cause the calibration system to: determine the velocity of blood flow within the vessel located near the ultrasonic Doppler sensor; adjust the velocity of blood flow within the vessel to a predetermined set point by inflating or deflating the inflatable cuff via the compressor; determine the pressure level to which the inflatable cuff is inflated or deflated to maintain the predetermined set point as measured via the pressure transducer, wherein the pressure level corresponds with a transmural blood pressure measurement for the wearer; and transmit the transmural blood pressure measurement to the host computer via the network, wherein the transmural blood pressure measurement is utilized to calibrate the continuous, non-invasive, blood pressure monitoring system to determine the cuffless blood pressure.

In one aspect, the pressure level can range from about 0 mmHg to about 300 mmHg.

In another aspect, the at least one ultrasonic transducer can be configured to measure variations in the diameter of the vessel in real time based on data associated with an emitted ultrasonic beam signal and a reflected ultrasonic beam signal. Further, the at least one ultrasonic transducer can include an emitter that emits the emitted ultrasonic beam signal and a detector that detects the reflected ultrasonic beam signal.

In still another aspect, the at least one ultrasonic transducer can include a piezoelectric micromachined ultrasound transducer (PMUT), a capacitive micromachined ultrasonic transducer (CMUT), a lead zirconate titanate (PZT) transducer, or a combination thereof or any type of sensor capable of measuring blood vessel diameter and velocity.

In one particular aspect, the piezoelectric micromachined ultrasound transducer can be a dual-electrode bimorph piezoelectric micromachined ultrasound transducer.

In yet another aspect, the piezoelectric micromachined ultrasound transducer can be operable at a resonant frequency ranging from about 2 MHz to about 10 MHz.

In a further aspect, the at least one ultrasonic transducer can be in the form of an array of ultrasonic transducers or as a single element transducer.

In one particular aspect, a protective material can be positioned over the array of ultrasonic transducers, and the protective material can include parylene, silicone, polydimethylsiloxane, or a combination thereof or any other appropriate packaging material suitable for the protective application.

In yet another aspect, the array of ultrasonic transducers or single element ultrasonic transducer can include a beam forming system for controlling beam steering angles.

In another aspect, the array of ultrasonic transducers can include at least four ultrasonic transducers.

In still another aspect, the power component can include a battery.

In one more aspect, the ultrasonic sensor patch can include an attachment means configured for ensuring contact to the wearer.

In another embodiment, a method for monitoring of blood pressure via a continuous, non-invasive, blood pressure monitoring system is contemplated by the present disclosure. The method includes: i) providing an ultrasonic sensor patch, the ultrasonic sensor patch comprising: at least one ultrasonic transducer located externally on a body part of the wearer and in a vicinity of a vessel; a power component; at least one processor; and a first memory including instructions capable of execution by the at least one processor; ii) emitting an ultrasonic beam signal from an emitter associated with the at least one ultrasonic transducer; iii) receiving a reflected ultrasonic beam signal by a detector associated with the at least one ultrasonic transducer; iv) determining, via the at least one processor, the diameter of the vessel using the emitted ultrasonic beam signal and the reflected ultrasonic beam signal; and v) determining, via the at least one processor, a cuffless blood pressure measurement for the wearer based on the diameter of the vessel.

In one aspect, the method can further include transmitting the cuffless blood pressure measurement to a host computer via a network.

In another aspect, the method can further include calibrating the continuous, non-invasive, blood pressure monitoring system by: i) providing a calibration system, the calibration system comprising: an inflatable cuff; at least one controller; a compressor in communication with the inflatable cuff; a pressure transducer; an ultrasonic Doppler sensor located externally on the body part of the wearer in the vicinity of the vessel and positioned proximal to the inflatable cuff; and a second memory including instructions capable of execution by the at least one controller; ii) inflating or deflating the cuff with the compressor; iii) measuring the pressure levels of to which the inflatable cuff with the pressure transducer as the inflatable cuff is being inflated or deflated in real time via the pressure transducer; iv) determining a velocity of blood flow within the vessel located near the ultrasonic Doppler sensor in response to the inflation of inflatable cuff; v) adjusting the velocity of blood flow within the vessel to a predetermined set point by inflating or deflating the inflatable cuff via the compressor; and vi) correlating determining the pressure level to which the inflatable cuff is inflated or deflated to maintain the predetermined set point as measured via the pressure transducer, wherein the pressure level corresponds with a transmural blood pressure measurement for the wearer.

In still another aspect, the method can include transmitting the transmural blood pressure measurement to the host computer via the network, wherein the transmural blood pressure is utilized to calibrate the continuous, non-invasive, blood pressure monitoring system to determine the cuffless blood pressure.

In yet another aspect, the calibration system can be disconnected from the continuous, non-invasive, blood pressure monitoring system once calibration has been completed.

In one more aspect, the ultrasonic transducer can be a piezoelectric micromachined ultrasound transducer (PMUT), a capacitive micromachined ultrasonic transducer (CMUT), a lead zirconate titanate (PZT) transducer, or a combination thereof, wherein the ultrasonic transducer, the ultrasonic Doppler sensor, or both are operable at a resonant frequency ranging from about 2 MHz to about 10 MHz.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

A full and enabling disclosure of the present disclosure to one skilled in the art, including the best mode thereof, is set forth more particularly in the remainder of the specification, including reference to the accompanying figures, in which:

FIG. 1 illustrates components of an example network-connected continuous non-invasive blood pressure monitoring system that can be utilized in accordance with various embodiments of the present disclosure;

FIG. 2A illustrates one example of an ultrasonic sensor that can be utilized in the continuous non-invasive blood pressure monitoring system according to various embodiments of the present disclosure;

FIG. 2B illustrates one example of cuffless ultrasonic sensor patch that can be utilized in the continuous non-invasive blood pressure monitoring system according to various embodiments of the present disclosure;

FIG. 3 illustrates example device interactions that can be utilized in accordance with various embodiments of the systems of the present disclosure;

FIG. 4 illustrates one example of a calibration system for measuring transmural blood pressure that can be utilized in the continuous non-invasive blood pressure monitoring system according to various embodiments of the present disclosure;

FIG. 5A illustrates an experimental setup of a tube sensing unit for testing an ultrasonic sensor in the form of a piezoelectric micromachined ultrasound transducer (PMUT) array using a pulse-echo testing technique;

FIG. 5B illustrates the echo signals from the tube sensing unit of FIG. 5A;

FIG. 6A is a graph illustrating the relationship between tube diameter and applied pressure in order to calibrate diameter changes under different static pressures;

FIG. 6B is a graph illustrating the linear relationship between the diameter and pressure after a calibration process;

FIG. 7A illustrates the experimental setup used to evaluate the influence of distance (d) on echo signals;

FIG. 7B is a graph illustrating the echo signals from tubes at varying distances (d);

FIG. 7C is a graph illustrating the decay of the echo signal as the distance (d) increases;

FIG. 8A illustrates the experimental setup used to evaluate the influence of lateral movements (Δ) on echo signals as the emitting angle (θ) changes;

FIG. 8B is a graph illustrating the echo signals from tubes at varying lateral distances (Δ);

FIG. 8C illustrates the directionality of the acoustic beam that influences the results shown in FIG. 8B;

FIG. 9A illustrates the experimental setup used to evaluate the influence of the in-plane angle (β) on echo signals;

FIG. 9B is a graph illustrating the echo signals from tubes at varying in-plane angles (p);

FIG. 10A illustrates the experimental setup used to evaluate the influence of the vertical angle (γ) on echo signals;

FIG. 10B is a graph illustrating the echo signals from tubes at varying vertical angles (γ);

FIG. 11A illustrates an ultrasonic sensor array that can be utilized in the continuous non-invasive blood pressure monitoring system according to various embodiments of the present disclosure;

FIG. 11B is a graph illustrating the blood vessel diameter over time as the blood pressure is varied as measured by the ultrasonic sensor array of FIG. 11A;

FIG. 12A illustrates an ultrasonic sensor array that can be utilized in the continuous non-invasive blood pressure monitoring system according to various embodiments of the present disclosure;

FIG. 12B illustrates the experimental setup for testing the ultrasonic sensor array of FIG. 12A; and

FIG. 12C is a graph illustrating the echo signals received by the ultrasonic sensor array of FIG. 12A with different steering angles.

Repeat use of reference characters in the present specification and drawings is intended to represent the same or analogous features or elements of the present invention.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present disclosure. Any of the features, components, or details of any of the arrangements or embodiments disclosed in this application are interchangeably combinable with any other features, components, or details of any of the arrangements or embodiments disclosed herein to form new arrangements and embodiments.

Generally speaking, the present invention is directed to a continuous, non-invasive, blood pressure monitoring system for determining a blood pressure measurement for a wearer. The system has accuracy close to that of an invasive catheter transducer system for continuous blood pressure measurement. The system includes an ultrasonic sensor patch, a processor, and a memory. The patch includes an ultrasonic sensor (e.g., transducer) located externally on a body part of the wearer and in a vicinity of a vessel that is configured to determine a diameter of the vessel. The patch also includes a power component. Further, the memory includes instructions that, when executed by the processor, cause the system to calculate a cuffless blood pressure measurement for the wearer based on the diameter of the vessel as determined by the transducer and transmit the cuffless blood pressure measurement to a host computer via a network. A method for continuous, non-invasive, blood pressure measurement is also provided.

Without intending to be limited by any particular theory, the present inventors have found that the features of the disclosed continuous, non-invasive, blood pressure monitoring system for determining a continuous blood pressure measurement for a wearer that has similar accuracy to an invasive catheter transducer system while being cuffless and non-invasive. Such a system allows for measurement of continuous blood pressure in ambulatory or home settings with greater patient compliance and without the need for invasive procedures to insert a catheter into the wearer or patient.

The continuous, non-invasive, blood pressure monitoring system can also include a calibration system that can be used in a clinic or hospital setting initially to calibrate the cuffless system for at home or ambulatory use. The calibration system can include an inflatable cuff, as well as a compressor with a pneumatic control valve that is in communication with the compressor and the inflatable cuff. The compressor can be used to inflate or deflate the cuff at a fast rate, and a pressure transducer is then used to measure the transmural pressure level in real-time to which the cuff is inflated. At the same time, an ultrasonic Doppler sensor can be located externally on the body part of the wearer and can be positioned proximal to the inflatable cuff. This sensor is configured to then determine a velocity of blood flow within the vessel, which is located near the ultrasonic Doppler sensor, in response to the inflation or deflation of the inflatable cuff. A controller then controls the blood vessel velocity to a set point of about 3 cm/sec. The system responds to inflate or deflate the cuff to maintain this velocity set point. As the control system operates, the transmural pressure is achieved as measured by the pressure transducer associated with the cuff. This control system then utilizes a memory that could be the same as the first memory above or a different, second memory, that executes instructions to measure the transmural blood pressure for the wearer based on controlling the velocity of blood flow within the vessel (e.g., brachial artery). This transmural blood pressure can then be used to calibrate the system to determine the cuffless blood pressure, as an initial pressure or P₀. In particular, per equation (1) below, the diastolic pressure, P_{d}, systolic pressure, Pₛ, and vessel stiffness, α, can be determined from the calibrated transmural initial pressure (P₀) data to calculate a continuous cuffless blood pressure.

$$P ( t ) = {P}_{d} * {e}^{α ( \frac{A ( t )}{{A}_{d}} - 1 )} \qquad ( 1 )$$

Further, α is calculated as follows, as shown in equation (2):

$$α = \frac{ln ( \frac{{P}_{s}}{{P}_{d}} )}{{( \frac{{D}_{s}}{{D}_{d}} )}^{2} - 1} \qquad ( 2 )$$

where the P_{d}, Pₛ, D_{d}, Dₛ, A_{d}, and α are the initial diastolic BP, initial systolic BP, initial diastolic diameter, initial systolic diameter, initial diastolic arterial cross-sectional area, and vessel rigidity coefficient, respectively. At the same time as the transmural initial pressure is being measured, (P₀), an ultrasonic sensor measures the initial vessel diameters that correlate with P₀ such that P_{d} and Pₛ correlate with D_{d}, and Dₛ, respectively. An initial diastolic cross-sectional area A_{d}, is calculated using D_{d}. Once the vessel rigidity coefficient is established, the calibration process is complete. The ultrasonic sensor remains on to measure real-time vessel diameter, D(t). The resulting pressure curve expression is shown below in equation (3):

$$P ( t ) = {P}_{d} * {e}^{α ( \frac{A ( t )}{{A}_{d}} - 1 )} = {P}_{d} * {e}^{α ( {( \frac{D ( t )}{{D}_{d}} )}^{2} - 1 )} \qquad ( 3 )$$

Finally, the resulting diameter curve can be converted to a pressure curve using this equation. Thereafter, the real-time cuffless blood pressure is determined via the ultrasonic sensor patch based on the vessel diameter as measured by the ultrasonic transducer within the ultrasonic sensor patch, the details of which are described in more detail below.

In any event, the pressure level to which the compressor can inflate the inflatable cuff can range from about 0 mmHg to about 300 mmHg, such as about 20 mmHg to about 250 mmHg, such as about 30 mmHg to about 240 mmHg, such as about 40 mmHg to about 230 mmHg.

Further, the ultrasonic sensor (e.g., transducer) can be a piezoelectric micromachined ultrasound transducer (PMUT), a capacitive micromachined ultrasonic transducer (CMUT), a lead zirconate titanate (PZT) transducer, or a combination thereof and can be in the form of a single transducer or an array of up to about 4 transducers, such as up to about 8 transducers, such as up to about 16 transducers, such as up to about 32 transducers or even higher. The ultrasonic transducer can also have beam forming system for controlling beam steering angles to allow for optimized signals to be emitted from the ultrasonic transducer to improve the accuracy of the system. Further, the ultrasonic sensor (e.g., transducer) associated with the ultrasonic sensor patch and the ultrasonic Doppler system associated with the calibration system can both be operable at a resonant frequency ranging from about 2 MHz to about 10 MHz, such as from about 2.5 MHz to about 9 MHz, such as from about 3 MHz to about 8 MHz.

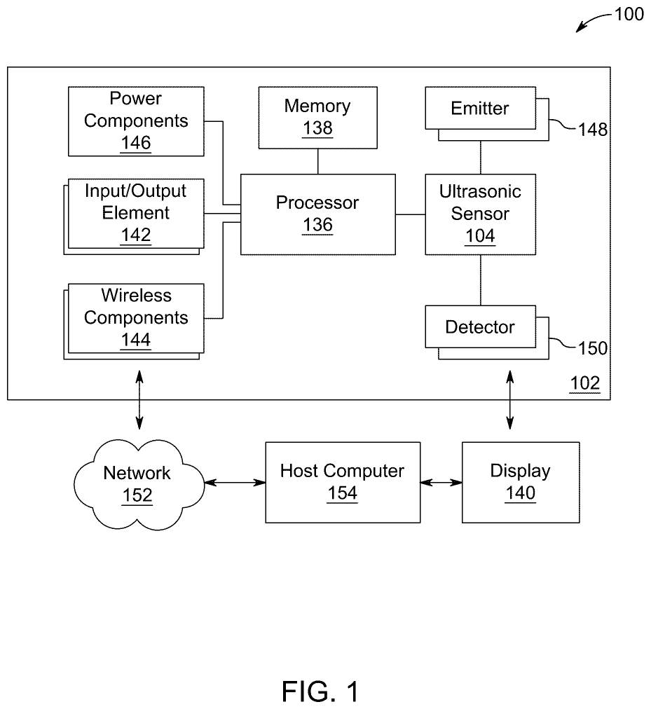

Referring now to FIGS. 1-12C, the particular components of the continuous, non-invasive, blood pressure monitoring system are shown in more detail. Specifically, FIG. 1 is an example continuous, non-invasive, blood pressure monitoring system 100. The system 100 can include a cuffless ultrasonic sensor patch 102 that can include one or more processors 136 coupled to memory 138. The ultrasonic sensor patch 102 can also be coupled to a display 140, a bus, one or more input/output (I/O) elements 142, and wireless networking components 144, among other such options. A display 140 and/or I/O devices 142 may be omitted in certain embodiments.

The I/O elements 142 can include one or more biometric sensors, optical sensors, barometric sensors (e.g., altimeter), and the like. The element(s) 142 can be used to track the location of the ultrasonic sensor patch 102 which can aid in determining wearer compliance over time. Upon determining an initial position of the ultrasonic sensor patch 102 (e.g., using GPS), the system 100 may track of the location of the ultrasonic sensor patch 102 by using the element(s), or in some instances, by using the orientation determining element(s) as mentioned above, or a combination thereof. The device also includes one or more power components 146, such as may include a battery operable for recharging through conventional plug-in approaches or through other approaches such as capacitive charging through proximity with a power mat or other such device. In some embodiments, the system 100 can include at least one additional input/output element 142 able to receive conventional input from a wearer. This conventional input can include, for example, a push button, touch pad, touch screen, wheel, joystick, keyboard, mouse, keypad, or any other such device or element whereby a user can input a command to the device from the ultrasonic sensor patch 102 itself, a host computer 154, or any other suitable personal computing device in communication with the ultrasonic sensor patch 102. These I/O elements 142 could even be connected by a wireless infrared or Bluetooth or other link as well in some embodiments.

Further, if included, a display 140 may provide an interface for displaying data, such as cuffless blood pressure readings and other metrics of the user. For example, the processor 136 may compute values for the physiological metrics monitored by one or more ultrasonic sensors 104 on the ultrasonic sensor patch 102. For instance, the one or more ultrasonic sensors 104 can be an ultrasonic transducer including an emitter 148 that emits ultrasonic signals as well as a detector 150 that receive ultrasonic signals reflected back from, for example, a vessel (e.g., brachial artery) that is in the vicinity of the ultrasonic sensor patch 102 that is positioned on a body party (e.g., limb such as an arm) of a wearer or patient. In an embodiment, the display 140 can be a part of a personal computer, tablet, mobile phone, laptop, or other suitable computing device. In other embodiments, the display 140 may be omitted and data detected by the ultrasonic sensor patch 102 may be transmitted using the wireless networking interface via near-field communication (“NFC”), Bluetooth, Wi-Fi, or other suitable wireless communication protocols over at least one network 152 to a host computer 154 for analysis, display, reporting, or other such use.

The memory 138 may comprise RAM, ROM, FLASH memory, or other non-transitory digital data storage, and may include a control program comprising sequences of instructions which, when loaded from the memory and executed using the processor 136, cause the processor 136 to perform functions described herein. The emitters 148 and detectors 150 may be coupled to a bus directly or indirectly using driver circuitry by which the processor 136 may drive the emitters 148 and obtain signals from the detectors 150. The host computer 154 may communicate with the wireless networking components 144 via one or more networks 152, which may include one or more local area networks, wide area networks, and/or the internet using any of terrestrial or satellite links. In some embodiments, the host computer 154 can execute control programs and/or application programs configured to perform some of the functions described herein.

In some embodiments, each emitter 148 can be individually controlled, or each detector 150 can be individually read out when multiple emitters 148 and detectors 150 are used, and in such embodiments, sensor data along several different ultrasonic signal paths can be collected. The control program can utilize the collected data to provide a more accurate estimation or blood pressure and/or other physiological metrics. In related aspects, the processor 136 and other component(s) of the system 100 may be implemented as a System-on-Chip (“SoC”) that may include one or more CPU cores that use one or more reduced instruction set computing (“RISC”) instruction sets, and/or other software and hardware to support the continuous, non-invasive, blood pressure monitoring system 100.

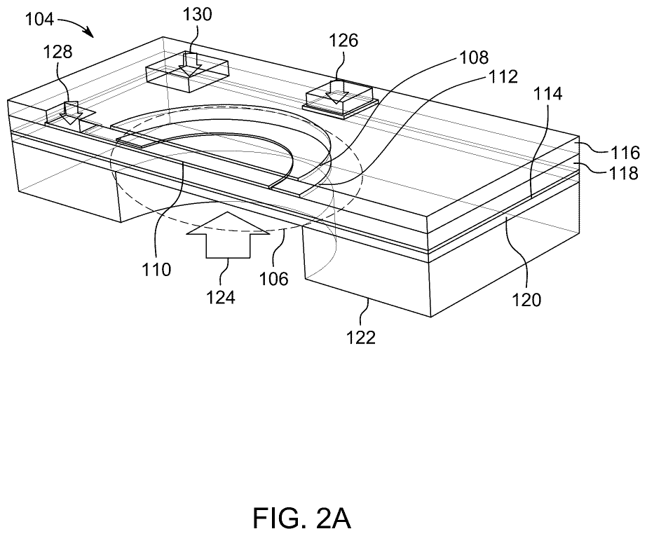

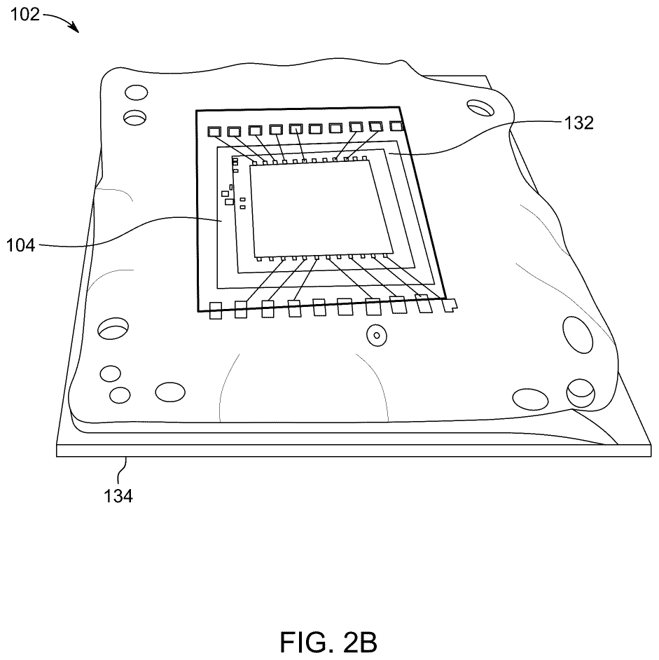

Turning now to FIGS. 2A and 2B, specific features of the ultrasonic sensor 104 (e.g., an ultrasonic transducer) and ultrasonic sensor patch 102 are shown in more detail. The ultrasonic sensor 104 can track real-time diameter variations of a blood vessel. Using the pulsed-echo technique or any suitable technique to transmit a high frequency acoustic pulse, its reflection (or echo) is received due to the variation in acoustic properties of the target medium (or impedance). This holds true for the interface between an artery wall and the blood moving within. Based on this knowledge, the Time-Of-Flight (TOF) interval between reflected echoes can be used to characterize the diameter of the blood vessel, which correlates to blood pressure (BP), P(t), as follows, and as described in more detail above:

$$P ( t ) = {P}_{d} * {e}^{α ( \frac{A ( t )}{{A}_{d}} - 1 )} \qquad ( 1 )$$

Where the P_{d}, A_{d}, and α are the diastolic BP, diastolic arterial cross-sectional area, and vessel rigidity coefficient, respectively. Diastolic BP and blood vessel stiffness are slow changing variables that can be determined very accurately (as accurate as an invasive catheter) with the transmural cuff device described in this application. Diastolic BP and blood pressure stiffness can also be determined less accurately through a standard spot check external blood pressure cuff or other methods such as the pulse wave velocity method, respectively. By monitoring the diameter in real-time using an ultrasonic sensor 104 as contemplated by the present disclosure, the real-time continuous blood pressure can be derived. The ultrasonic sensor 104 can be a transducer in the form of a micromachined ultrasound transducer (MUT), where the active element is a piezoelectric diaphragm that vibrates when AC current is applied, generating ultrasonic pressure (transmitting ultrasonic waves). Reflected ultrasonic waves (echoes) that are emitted and that reach the surface of MUTs cause mechanical deformation of the diaphragm, generating electrical signals that are then received by a detector in the form of an ultrasonic echo. In comparison to traditional ultrasound transducers, MUTs offer several advantages: wide bandwidth, miniaturization, large arrays of transducers, and on-chip integration with microelectronics. The two primary types of MUTs are capacitive MUTs (CMUTs) and piezoelectric MUTs (PMUTs). CMUTs use high DC bias voltages of more than 100V to achieve nanometer resolution. In contrast, PMUTs are driven by piezoelectric force without the DC bias voltage requirement and have favorable bandwidth, cost, and yield. These attributes contributed to the selection of PMUTs for this project.

FIG. 2A illustrates one potential MUT for the ultrasonic sensor 104 of the present disclosure in the form of a dual-electrode biomorph PMUT design, which can be incorporated into the ultrasonic sensor patch 102 of FIG. 2B, although it is to be understood that any suitable MUT or even a lead zirconate titanate (PZT) transducer can be used for the ultrasonic sensor 104 of the present disclosure. The dual-electrode biomorph design PMUT as shown in FIG. 2A can increase the acoustic pressure and resulting echo signals by the differential driving mechanism. A more advanced approach to further enhance the acoustic output is to alleviate the diaphragm 106 boundary from fully clamped to “pinned” boundary. By alleviating the boundary constraints, which restrict vertical movements of the diaphragm 106 under piezoelectric excitation, the total pressure output is expected to increase to levels that are 12 to 16 times higher than conventional PMUT designs. The PMUT fabrication process involves using AlN as the piezoelectric layer(s). The fabrication process can include a sputtering of an AlN/Mo/AlN stack with thicknesses of 200 nm, 100 nm, 1000 nm, respectively, onto a substrate 122. The first AlN layer serves as the backside deep reactive ion etching (DRIE) stop layer 120, while the Mo layer is the bottom electrode 114 and the second AlN layer is the bottom active layer, which can be referred to as the first active piezoelectric layer 118. A thin SiO₂ layer is deposited and followed by the deposition of the second Mo layer (100 nm) for the middle electrode 112. The SiO₂ layer is used to prevent the breakdown of the stability of the ultrasonic sensor 104 by preventing the possible diffusion of the middle Mo electrode 112 to the bottom AlN layer along the crystalline defects. The middle Mo and the silicon dioxide barrier layers are defined and etched using SF₆ and O₂ plasma etching. The second 1000 nm-thick AlN active layer or second piezoelectric active layer 116 is then deposited together with a second 50 nm-thick SiO₂ barrier layer. The top Mo electrode layer 108 is then deposited and patterned. Afterwards, the via structure included the via 126 to the inner mid-electrode 110, the via 128 to the outer mid-electrode 112, and the via 130 to the bottom electrode 114 are etched in a two-step process, and etch-through hole 124 is also created. The via etching to the bottom and middle electrodes 110, 112, and 114 are conducted simultaneously by reactive ion etching (RIE). The final via etching ends by a final wet etching of AlN. An initial prototype has been designed, fabricated, and packaged with a protective material 132 that can include polydimethylsiloxane (PDMS), parylene, silicone, or a combination thereof, and a tape to secure the wiring channel connections on the PMUT printed circuit board (PCB) as shown in FIG. 2B. Further, attachment means 134 (e.g., tape, adhesive, etc.) can be used to secure the ultrasonic sensor patch 102 to a wearer and/or to secure various components of the ultrasonic sensor patch 102 in place.

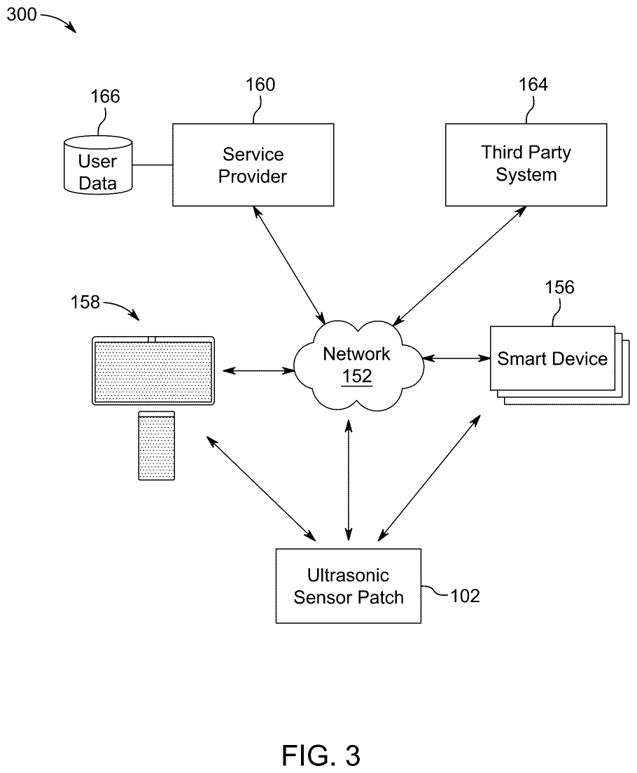

Regardless of the particular type of ultrasonic sensor patch 102 or ultrasonic transducer 104 utilized, the ultrasonic sensor patch 102 can be connected to various components for data monitoring and review as shown in the system overview 300 of FIG. 3. In at least some embodiments, cuffless blood pressure related information determined for a wearer utilizing the system 100 can be presented to, or provided for, the wearer to a medical professional, the wearer, or others in a number of different ways using a number of different devices or communication channels. This can include raw data for analysis or the results of such analysis, among other such options. In at least one embodiment, blood pressure data 166 may be collected or determined using the ultrasonic sensor patch 102 associated with a wearer, as illustrated in the system overview 300 of FIG. 3. In at least some embodiments, such data may also be collected by one or more smart devices 156 associated with the wearer. Such smart devices can include any device able to collect or determine data that may be useful in blood pressure analysis and provide that data 166 in some way to another computing device, such as through a wired or wireless connection, where that connection may be direct, across at least one network 152, or through one or more other devices or channels. In at least one embodiment, this data may be collected for analysis by a user computing device 158, such as a tablet or desktop computer running an application with sleep analysis functionality. In at least some embodiments, at least some of this data 166 can be provided to a service provider system 160 associated with the ultrasonic sensor patch 102 or health monitoring software. In some embodiments, a wearer may instead subscribe to a service offered by this service provider system 160, which can then receive data and provide sleep-related analysis or recommendations. Some embodiments may also utilize a third party system 164 or service for at least a portion of this data collection or analysis, or to collect related data useful in the analysis. For example, this third party system 164 might provide blood pressure data for other individuals, updated blood pressure analysis, and so on. In some embodiments the third party system 164 may be associated with one or more of the smart devices 156, and can provide data that is collected by those devices and provided to the third party system. For example, a smart alarm system that can provide information about low or high blood pressure readings other such data may provide that data to the third party system 164, and then the third party system may provide at least some of this data to a service provider 160 for analysis, if permitted by the wearer, the wearer's medical professional, and under local privacy laws, etc.

Such an approach has various advantages, as multiple types of devices 102, 156, and 158 can be used to collect data that may be relevant to the sleep of an individual. This data can then be transmitted over at least one network 152 to a device, system, or provider that is able to aggregate and analyze this data to determine various blood pressure-related metrics and other such information. Such an approach is also beneficial because the devices used to collect and transmit this data do not need to have substantial processing or memory capacity, since the majority of the data analysis and processing can be performed by the remote system, service, or device.

Results of such blood pressure analysis, or other health or state analysis, may also be provided back to any of these or other such systems, devices, services, or providers. For example, data or instructions may be provided to the smart devices 156 to adjust a state or perform a task. In some embodiments data or instructions might be provided to a central system or device, such as a management device, which may then send individual instructions to relevant devices to perform specific tasks or make specific changes. Data or instructions may be provided to the ultrasonic sensor patch 102 being worn by a user to provide prompts or recommendations to the user that should be relatively instantaneously received. Data or instructions can also be provided to a user computing device 158 for presentation to a user, such as to provide updated blood pressure information, changes in blood pressure metrics, recommendations for improving blood pressure, and so on.

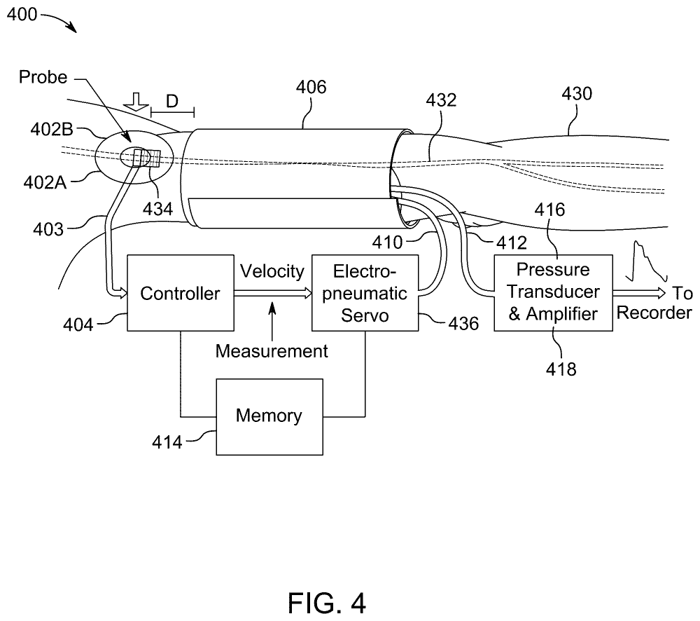

Turning now to FIG. 4, the cuffed calibration system 400 that can be used in conjunction with the continuous, cuffless, non-invasive blood pressure monitoring system 100 of the present disclosure will be described in more detail. The noninvasive calibration method uses one or more ultrasonic Doppler sensors 402A connected to a controller 404 via a connection 403, where the controller 404 and a second memory 414 are utilized to execute instructions to an electro-pneumatic servo or pneumatic valve 436 to control the pressure in the cuff 406 that is placed around a body part 430 (e.g., a limb such as an arm) of the wearer and utilizes transcutaneous ultrasound Doppler techniques to detect blood flow within a vessel 432 (e.g., an artery such as the brachial artery) as shown in FIG. 4.

In some embodiments contemplated by the present disclosure, the pressure in the cuff 406 is controlled by a fast electropneumatic servo or pneumatic valve 436 and monitored by a pressure transducer 416 that is coupled to the cuff 406 via a connection 412, where a pressure transducer 416 can optionally be used and configured to monitor internal pressure of the vessel 432. Additionally, a tube 410 is connected to the cuff 406 to inflate the cuff 406 with air when connected to the servo or pneumatic valve 436 via a tube 410. The blood flow velocity in the vessel 432 is assumed to be proportional to the blood flow and is detected by an ultrasonic Doppler sensor 402A. It has been shown that the detection of blood flow velocity continuously by using one or more ultrasonic Doppler sensors 402A or an array thereof that emit ultrasonic beams 434 and that are positioned proximal to the cuff 406 and located a distance D from the cuff 406 is a more direct indicator of arterial opening than those from the state-of-art Korotkoff sounds of oscillations in standard blood pressure cuff pressure measurements. The distance D can range from about 1 millimeter to about 250 millimeters, such as from about 5 millimeters to about 200 millimeters, such as from about 10 millimeters to about 150 millimeters. Further, an amplifier 418 can be used to amplify signals from the pressure transducer 416, so that the signals being correlated and/or calibrated to a blood pressure reading can be visualized and analyzed according to methods contemplated by the present disclosure.

Generally, the cuffed calibration system 400 can utilize a pulsed-wave ultrasonic Doppler sensor, or any other suitable Doppler transmit/receive method for 402A capable of deep and wide ultrasound beam signals. For example, the ultrasonic Doppler sensor can be a 3 MHz resonant frequency single element PZT (lead zirconate titanate) transducer. The calibration system 400 can include architecture that is responsible for generating ultrasound transmission waves to the ultrasonic Doppler sensor 402A and receiving the reflection from the sensor 402A, 402B. This pulse/echo event is governed by the blood flow Doppler effect, which is controlled by the controller 404. Preliminary results show pressure data produced is comparable to an invasive pressure sensor. Further, it should be understood that an additional ultrasonic Doppler sensor 402B can be utilized to measure vessel diameter as opposed to flow to further enhance the accuracy of the calibration system 400.

The present disclosure is further described in the following Examples, which do not limit the scope of the disclosure described in the claims.

### Example 1

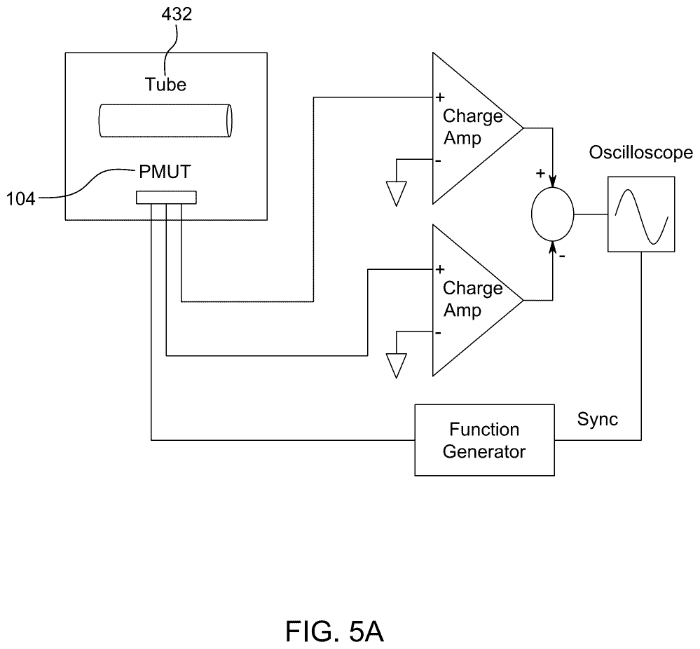

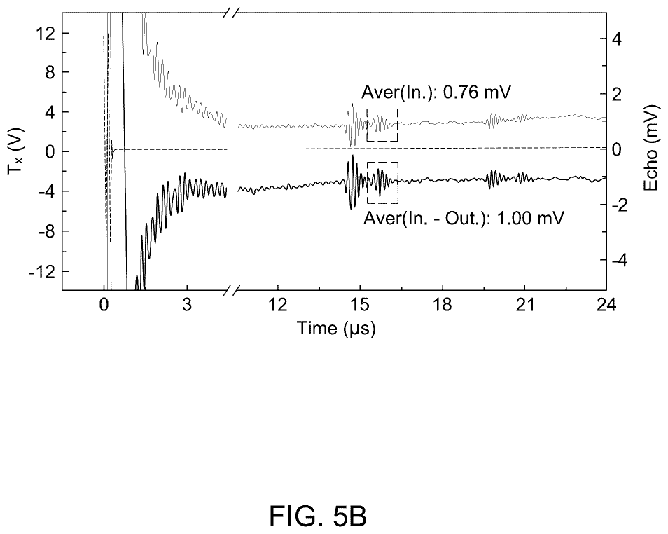

In Example 1, the ultrasonic sensor 104 of the ultrasonic sensor patch 102 of the present disclosure has been characterized experimentally using a pulse-echo testing technique in a mineral oil batch with the setup as shown in FIG. 5A with an in vitro vessel 432. The diameter of the vessel 432 was determined by monitoring the locations of the anterior and posterior wall of the blood vessel and the blood pressure is calculated by using equation (1) set forth above. Meanwhile, FIG. 5B displays echo signals from the tube sensing result, where the top/upper curve represents the signal obtained by averaging the inner electrodes of the PMUTs only, and the bottom/lower curve is the signal from averaging the difference between the inner and outer electrodes. It is found that there are four echoes from both sides of anterior and posterior walls of the tube as expected. The signal from the outer side of the anterior wall is at 14 μs, which corresponds well to the 10 mm distance in the real experimental setup. The inner sides of the anterior and posterior walls are crucial in extracting the blood vessel diameter. As such, the amplitude of the inner side of the anterior wall is marked in the figure. The convex shape of the inner side wall inherently generates a smaller signal due to the divergence of the reflecting ultrasound wave. Furthermore, the receiving amplitudes of the inner and (inner-outer) electrode are 0.76 mV and 1.00 mV, respectively, indicating the outer electrode contributes a 33% amplitude as compared to that of the inner electrode. The performance reduction from the outer electrode is attributed to two factors: (1) the backside hole sidewall edge of PMUTs is not a perfect circle due to the DRIE etching process, which could alter the diaphragm's boundary condition; and (2) the fabricated diaphragms have a larger radius of 32 μm as compared to the designed value of 29 μm. Consequently, the outer electrode has a portion of its region located within 70% of the new enlarged radius, which negatively influences the output amplitude.

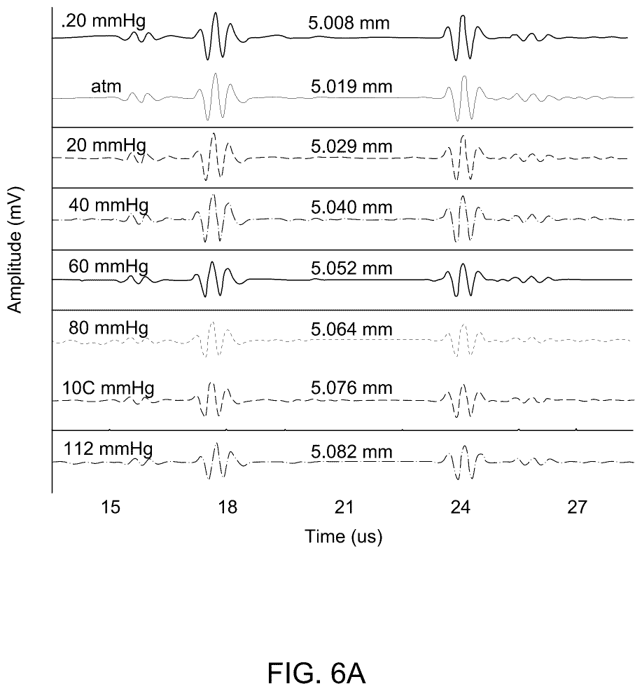

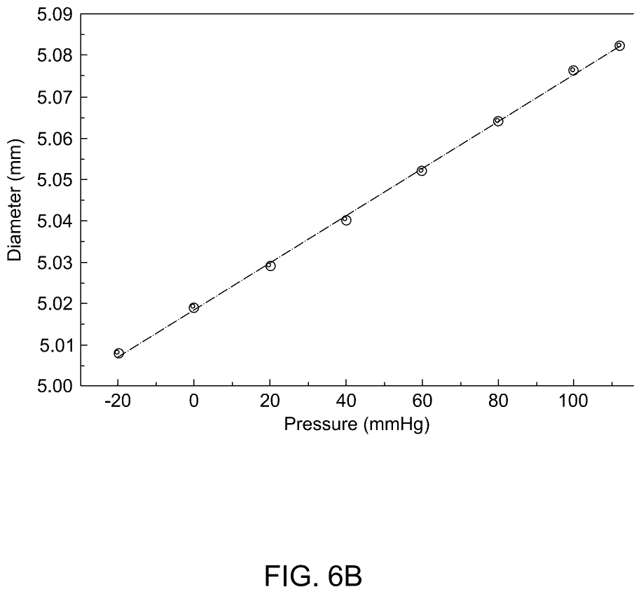

The relationship between the diameter of the tube and applied pressure is characterized by calibrating the diameter changes under different static pressures. The pressure range between −20 mmHg to 112 mmHg is tested and the result shows approximately 100 μm in diameter variation. FIG. 6A displays the waveforms corresponding to different pressures. As shown, a larger time interval is observed when higher pressure is applied. Next, a calibration process is conducted based on these waveforms, and the correlation is illustrated in FIG. 6B. A linear relationship between the diameter and pressure is found for small diameter changes between 20 mmHg and 112 mmHg.

### Example 2

In real-world applications, it is difficult to consistently maintain a perfect alignment between the PMUT sensor and the target artery as the sensor may move its position during the tests or over a long period of time. Specifically, during the ultrasound Doppler servo method tests, we often encountered a very long delay time to try to find the right position of the blood vessel. This problem may influence the signal quality and should be addressed by schemes such as mechanical fixation or acoustic beam steering. Here, the influences due to the variations of the distance d, lateral shift Δ, in-plane angle β, and vertical angle γ are analyzed as preliminary results.

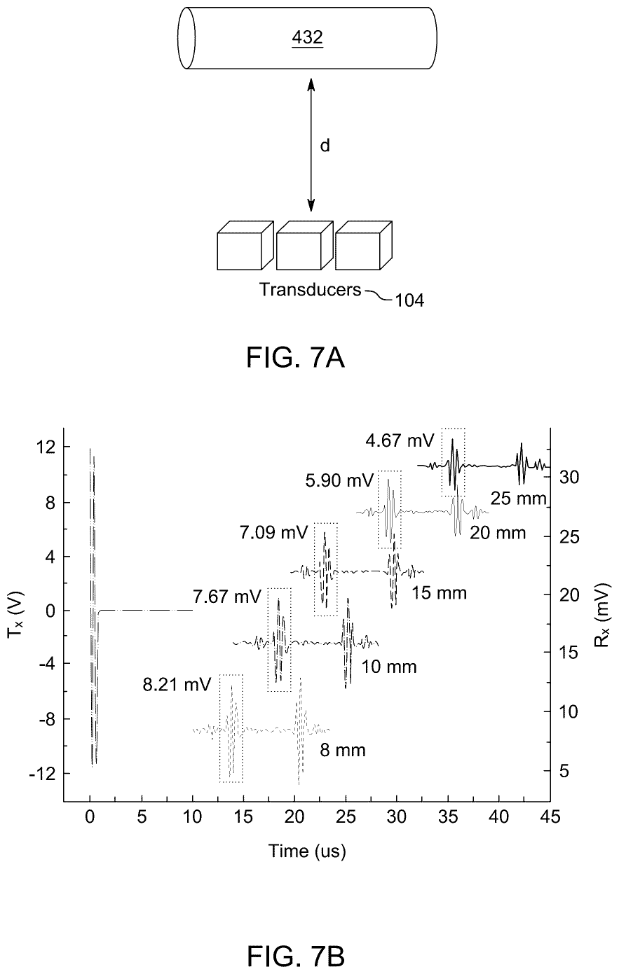

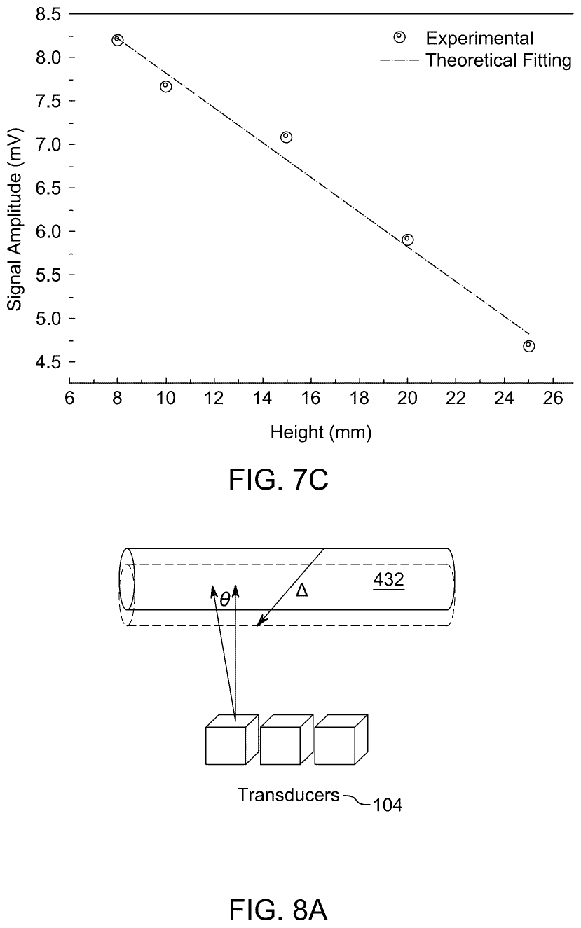

FIG. 7A illustrates the experimental setup used to evaluate the influence of distance d on echo signals. The same tube is positioned at different distances relative to the PMUTs. The excitation pulse signal has two 24 Vₚₚ rectangular pulse cycles, and the receiving signal is collected via the receiving channels. FIG. 7B shows that the echo signal decreases from 8.21 mV to 4.67 mV as the distance increases from 8 mm to 25 mm. The decay can be modeled by the exponential decay formula as shown in FIG. 7C.

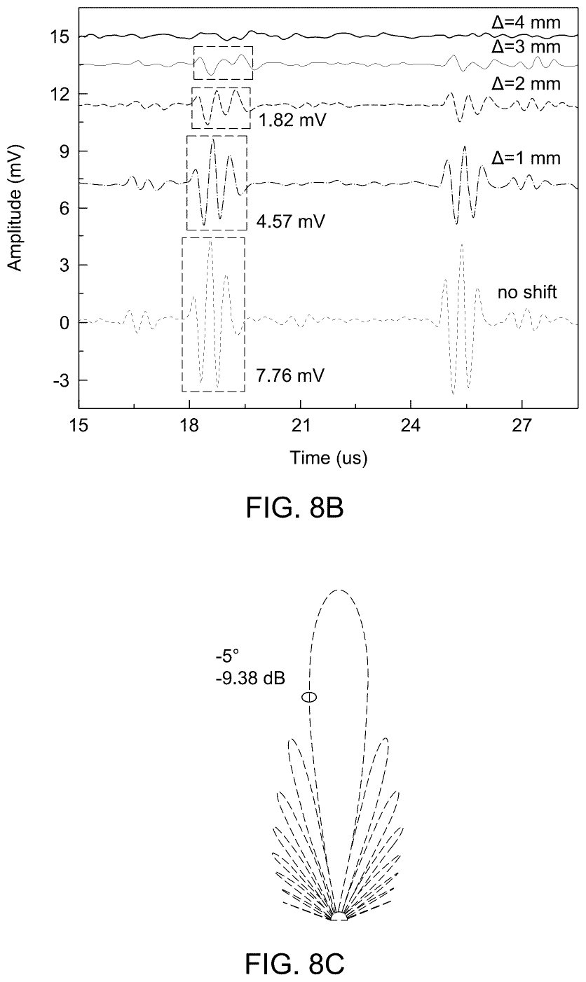

FIG. 8A shows the setup to evaluate the influence of the lateral movements of PMUTs with respect to the tube. The tube is placed in parallel to the PMUT array while being moved out of the plane and the corresponding signal is measured as shown in FIG. 8B. It is observed that the signal drops quickly as the lateral movement increases. A lateral movement of 1 mm results in a signal drop of 41%. In fact, it becomes difficult to identify the echo peak for a lateral movement of 2 mm. This result can be attributed to the directionality of the acoustic beam, as depicted in FIG. 8C. A lateral shift of 1 mm generates an emitting angle θ of 5° from the PMUTs to the tube, which corresponds to an amplitude of −9.38 dB or 34% reduction to the peak value. As such, the ultrasound wave emitted from or received from this angle would not be efficient to generate strong signals. To correct this possible lateral movement issue in real applications, one possible approach is to use the beamforming technology to redirect the acoustic beam to the tube.

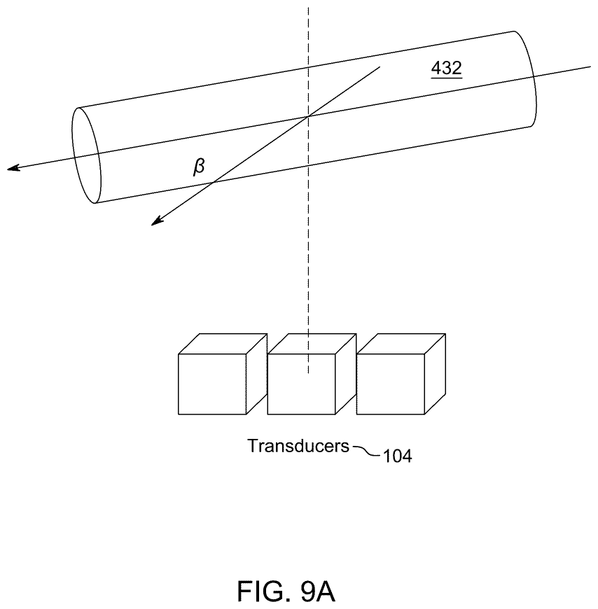

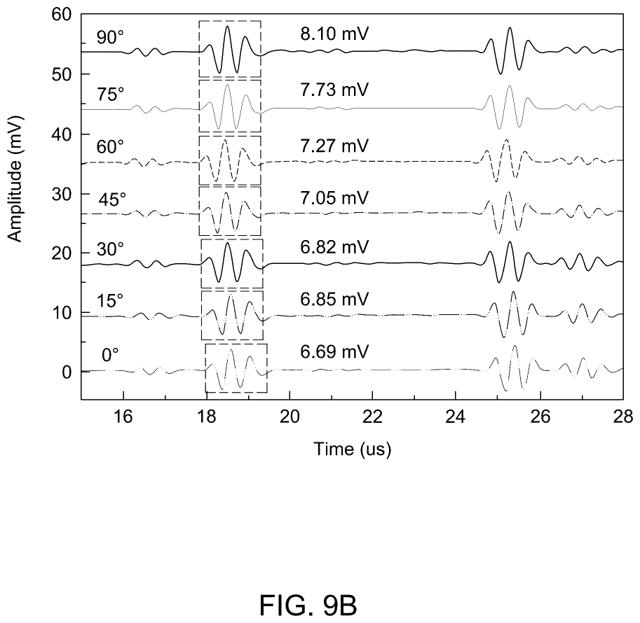

FIG. 9A is the setup to evaluate the influence of the in-plane angle, β, ranging from 0° to 90° and the echo signals. The tube is placed in a plane in parallel to the PMUTs surface plane at a distance away as shown. The zero-degree line is defined as the direction perpendicular to the line-shape of PMUTs as illustrated. The corresponding echo signals are shown in FIG. 9B. It is observed that the receiving signal increases 21% as β increases from 0° to 90°. As the tube is in-parallel with the PMUT array to align with the line-shape of PMUTs, this results in the better utilization of the acoustic beam and stronger echo signals. This suggests that it is important to maintain a larger overlap area between the ultrasonic sensor patch/ultrasonic sensor and the vessel (e.g., artery).

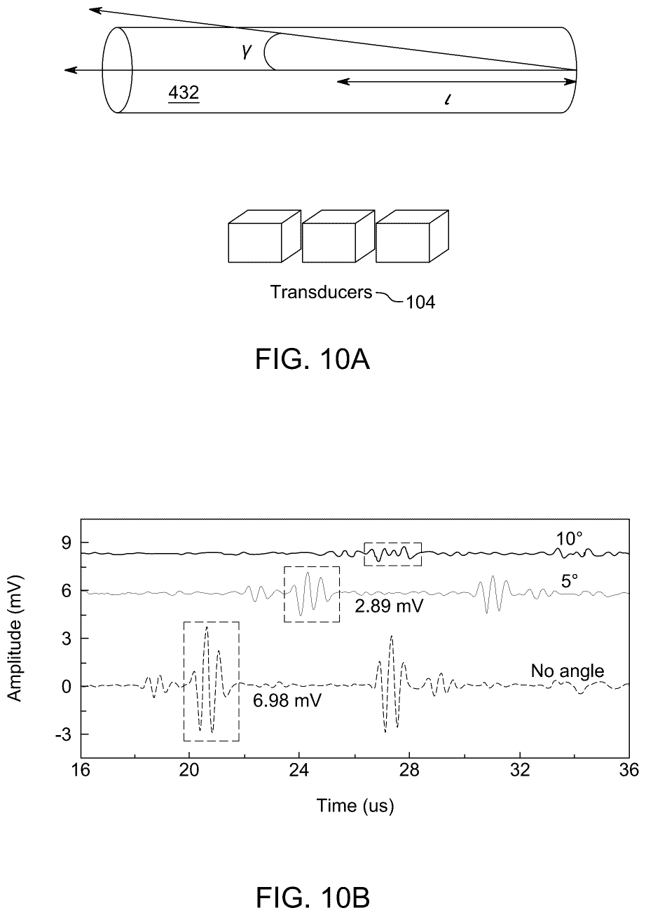

FIG. 10a depicts the experimental setup used to evaluate the influence of the vertical angle γ on the echo signals. The tube is placed in parallel to the line-shape arrangement of the PMUTs at a distance away as illustrated and this is defined as γ=zero degree. By fixing the right starting point at a distance of I (30 mm in this case) to the center of the PMUTs array, the tube is rotated with and angle, γ, from 0° to 10°, along the plane vertical to the surface of the PMUTs array. The results are shown in FIG. 10B. It is found that the vertical angle has a significant impact on the signal quality as the echo-signal drops by almost 60% with a 5° vertical tilt. As the tube is no longer in-parallel with the PMUT array, there is a significant time delay for the reflected ultrasound to reach individual PMUT element to results in the reduction of the signal strength (similar to the effect of beam forming without the optimization process for reduced amplitudes).

Overall, among the four factors, the lateral movements and vertical angles have significant influences on the signal strength. These issues are can be adjusted by the beam-forming technology to adjust the focus of the acoustic beam to a specific position in the 3D space. These results show the necessity for the proposed PMUTs array structure for beam-forming operation to increase sensing signals and to seek the location of the blood vessel for wearable blood pressure measurements by normal people without medical training.

### Example 3

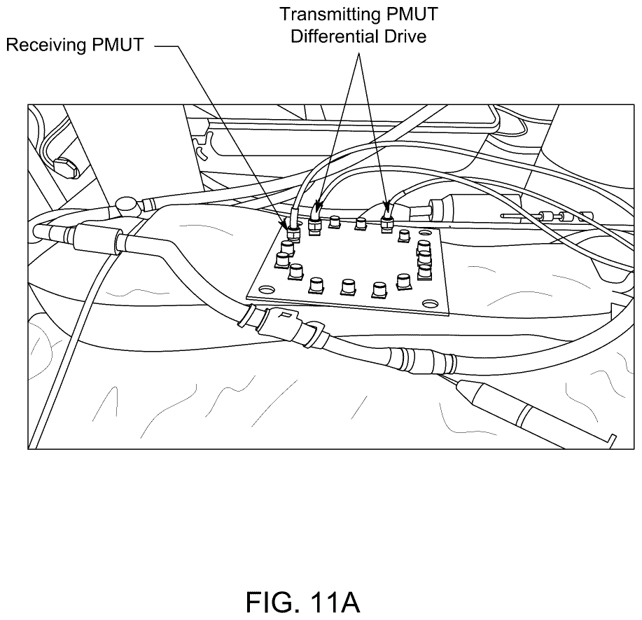

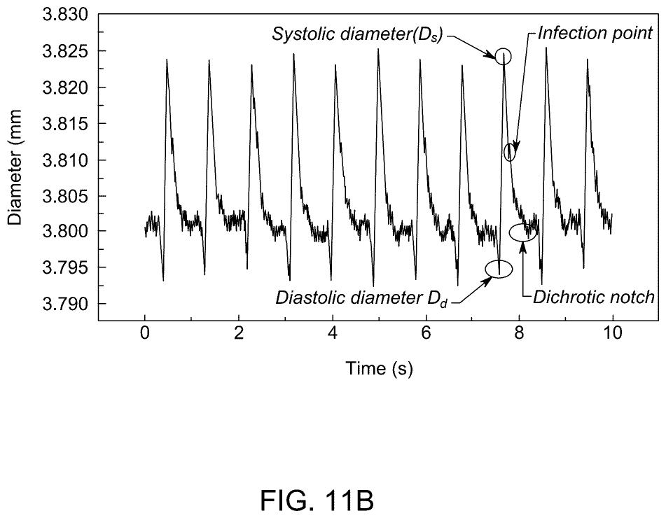

In Example 3, prototype PMUTs were packaged and placed on the phantom arm with a PCB board as shown in FIG. 11A. Initial testing results showed lots of noises due to limited signal strength, encapsulation package, and non-matching amplifier gain. Improvements include using an alternative material of Parylene to cover the whole PMUT surface to minimize acoustic signal losses and silicone as a protection layer to prevent wire breakage and the improved PMUT system can detect dynamic vessel diameter as shown in FIG. 11B.

The characteristics of measured diameter range correlate to the peak of systolic pressure (SP), diastolic pressure (DP) and the dicrotic notch well. The system can correlate echo timing with anterior and posterior wall diameter measurements successfully along various brachial arterial vessel locations of the phantom. Various flow rates were also tested ranging between 40-100 beats/min at various locations along the brachial artery phantom and the optimal data were collected at 60 beats/min from the 3.5 MHz resonant frequency PMUT.

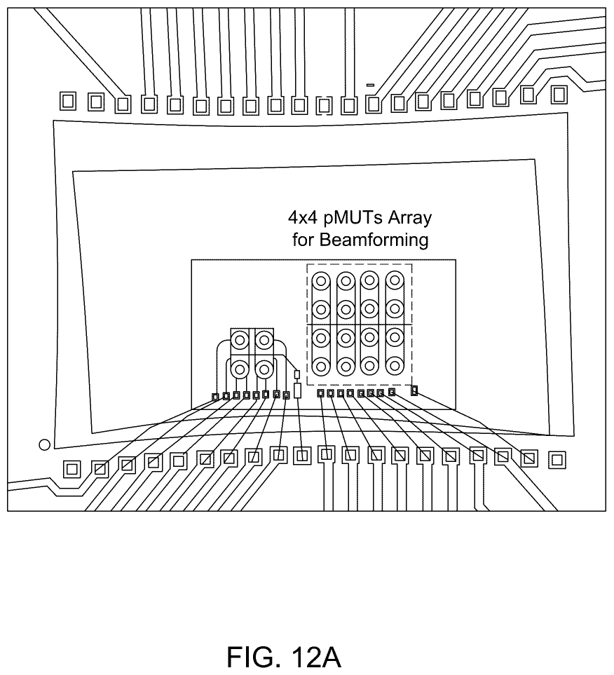

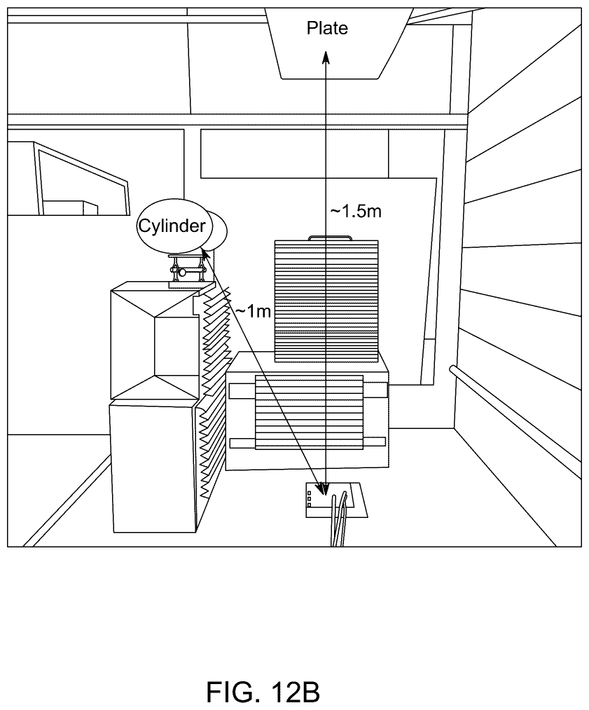

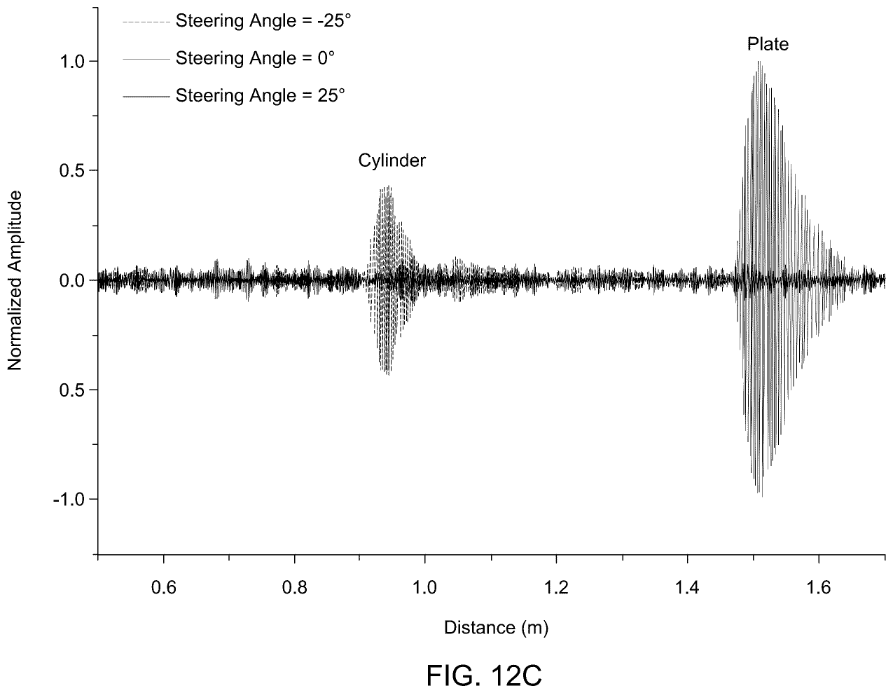

In general, beam-forming is a signal processing technique in sensor arrays for directional signal transmission or reception such that that signals at particular positions experience constructive interference while others experience destructive interference. For example, receiving beam-forming has been demonstrated by using a 4×4 PMUT array for 3D space sensing application in FIG. 12A.

Specifically, the pitch size between PMUT elements is designed to be 1.5 mm, which is close to the half wavelength of the ultrasound signals (A/2=1.29 mm) in air at 133 kHz, in order to suppress the grating lobes. PMUT elements in each column are connected to act as a single channel, which results in a total of 4 channels for the 2D ultrasound transmission beamforming scheme. The PMUT channels are excited by 15-cycle 20 Vₚₚ pulses at 133 kHz with designated phase delays to control the ultrasound transmission beam forming angles. Furthermore, the inner electrodes are excited for transmission and outer electrodes are used for the receiving function to have the transmission and receiving functionalities on the same chip. By using a larger PMUTs array with more channels and a multi-channel circuit for transmitting and receiving signals, the angular resolution can be further increased. FIG. 12B shows the experimental setup in air. A plate is fixed about 1.5 meters above the PMUT chip and a cylinder is placed around 1 meter away from the chip with an angle of about 20°. Foam absorbers are used to eliminate the undesired echo signal reflected by the box which is used for lifting the iron pole to support the plate structure. A transmission beamforming scheme is utilized to demonstrate the space imaging capability. The ultrasound beam is steered between −45° to 45° laterally with a 5° step.

FIG. 12C shows the received echo signals of the PMUTs array with three different steering angles after the post-processing with a Butterworth bandpass filter in MATLAB R2018b. The processed signals show clear echoes when the beam is steered toward the cylinder and the plate, whereas no echo signal is observed when the beam is steered in directions without any object.

While various embodiments of the present disclosure have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

In summary, preliminary evaluations have shown that accurate detection of BP in vitro is dependent upon SNR, resonant frequency, PMUT density and aperture size. The ultimate design of the sensor focuses on: (1) the number of PMUT elements to balance the ultrasonic pressure output and the size of the chip; (2) the resonant frequency to optimize axial resolution and field of view; and (3) transmission beam-forming schemes. A sensor array with high acoustic pressure and high sensitivity can increase the SNR to reduce measurement errors and transmitting beam-forming can improve the signal strength by concentrating/increasing the acoustic pressure at specific locations. On the other hand, receiving beam-forming as demonstrated in FIGS. 12A-12C can increase the sensing signals and can be utilized. By leveraging theoretical calculations, frequency and acoustic simulations, optimal PMUT designs both beam-forming schemes and maximized SNR can be achieved.

Sensor coating/interface materials that meet biocompatibility and performance requirements for a wearable system are also important to meet packaging and practical usage requirements. Previously, PDMS (polydimethylsiloxane) has been used in many PMUT devices for packaging/interface functions, but other biocompatible materials such as the ultrasonic gels commonly used in traditional ultrasonic systems can also be utilized as the interface material. A recent report also explored a couplant made of a soft, tough, anti-dehydrating, and bio-adhesive hydrogel-elastomer hybrid as the interface for ultrasound images of 48 hours which will be studied with measurements both before and after the application of the packaging/interface materials. Theoretically, it is expected that the coating layer could induce a small resonant frequency change which can be easily calibrated.

The fabrication and packaging/interface of an optimized sensor whose fundamental acoustic performances will be conducted with hydrophone measurements with respect to frequency and acoustic simulations is also considered. Sensor performance can be evaluated with the pulsed wave Doppler scheme for calibration with the cuff and the diameter variation scheme for blood pressure measurements without the cuff. A phantom system (similar to FIG. 4) will be utilized to test the performances of the ultrasound system in terms of: (1) calibration process with the cuff; and (2) pressure measurements based on blood vessel diameter variations without the cuff. A pressure sensor will be used in the simulated blood vessel to measure the “true pressure”. Measurements will be collected in two-minute increments across ten consecutive, fifteen-minute measurement windows.

It is also important to have beam-forming schemes to increase the sensing signals and to perform automatic search function for the blood vessel. We will test the system with and without using the beam-forming schemes to compare the results. Theoretically, we anticipate that without the beam-forming scheme, it will take several trial tests to get meaningful signals for pressure measurements.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the present disclosure, but instead as merely intended to highlight alternative or additional features that may or may not be utilized in a particular embodiment of the present disclosure. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments. For instance, when a plurality of ranges are provided, any combination of a minimum value and a maximum value described in the plurality of ranges are contemplated by the present invention. For example, if ranges of ‘from about 20% to about 80%’ and ‘from about 30% to about 70%’ are described, a range of ‘from about 20% to about 70%’ or a range of ‘from about 30% to about 80%’ are also contemplated by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more” or ‘at least one’ and indefinite articles such as ‘a’ or ‘an’ (e.g., ‘a’ and/or ‘an’ should typically be interpreted to mean ‘at least one’ or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the terms ‘about,’ ‘approximately,’ or ‘generally.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches. As used herein, the terms ‘about,’ ‘approximately,’ or ‘generally,’ when used to modify a value, indicate that the value can be raised or lowered by 5% and remain within the disclosed embodiment.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

### PRIVACY

It should be understood that individual health data collection such as blood pressure measurements may include sensitive and confidential information relating to a user, such as predisposition for hypertension, heart disease, etc. Accordingly, and further to the descriptions above, all data and related information acquired using the products or end products of the aforementioned systems and methods will be kept private and confidential. Thus, it should not be construed that any information discovered or inferred from the use of the products or end products produced using the aforementioned systems and methods will be improperly used or published. For example, information acquired from the devices and systems contemplated by the present disclosure may be treated so that no person without express or implied consent is capable of accessing said information. Thus, the information acquired from the devices and systems contemplated by the present disclosure may be kept confidential and access to the information may be controlled exclusively by the user of the devices and systems contemplated by the present disclosure whose information is determined by using the devices and systems contemplated by the present disclosure.

What is claimed is:

1. A continuous, non-invasive, blood pressure monitoring system for determining a blood pressure measurement for a wearer, the system comprising:
an ultrasonic sensor patch, the ultrasonic sensor patch comprising:
at least one ultrasonic transducer located externally on a body part of the wearer and in a vicinity of a vessel, the at least one ultrasonic transducer configured to determine a diameter of the vessel;
a power component;
at least one processor; and
a first memory including instructions that, when executed by the at least one processor, cause the continuous, non-invasive blood pressure monitoring system to:
calculate a cuffless blood pressure measurement for the wearer based on the diameter of the vessel as determined by the at least one ultrasonic transducer; and
transmit the cuffless blood pressure measurement to a host computer via a network.

2. The system of claim 1, further comprising a calibration system, the calibration system comprising:
an inflatable cuff;
at least one controller;
a compressor in communication with the inflatable cuff and configured to inflate or deflate the cuff;
a pressure transducer configured to measure pressure levels to which the inflatable cuff is inflated or deflated in real time;
an ultrasonic Doppler sensor located externally on the body part of the wearer in the vicinity of the vessel and positioned proximal to the inflatable cuff, the ultrasonic Doppler sensor configured to determine a velocity of blood flow within the vessel in response to the inflation or deflation of the inflatable cuff; and
a second memory including instructions that, when executed by the at least one controller, cause the calibration system to:
determine the velocity of blood flow within the vessel located near the ultrasonic Doppler sensor;
adjust the velocity of blood flow within the vessel to a predetermined set point by inflating or deflating the inflatable cuff via the compressor;
determine the pressure level to which the inflatable cuff is inflated or deflated to maintain the predetermined set point as measured via the pressure transducer, wherein the pressure level corresponds with a transmural blood pressure measurement for the wearer; and
transmit the transmural blood pressure measurement to the host computer via the network, wherein the transmural blood pressure measurement is utilized to calibrate the continuous, non-invasive, blood pressure monitoring system to determine the cuffless blood pressure.

3. The system of claim 2, wherein the pressure level ranges from about 0 mmHg to about 300 mmHg.

4. The system of claim 1, wherein the at least one ultrasonic transducer is configured to measure variations in the diameter of the vessel in real time based on data associated with an emitted ultrasonic beam signal and a reflected ultrasonic beam signal.

5. The system of claim 4, wherein the at least one ultrasonic transducer includes an emitter that emits the emitted ultrasonic beam signal and a detector that detects the reflected ultrasonic beam signal.

6. The system of claim 1, wherein the at least one ultrasonic transducer comprises a piezoelectric micromachined ultrasound transducer (PMUT), a capacitive micromachined ultrasonic transducer (CMUT), a lead zirconate titanate (PZT) transducer, or a combination thereof.

7. The system of claim 6, wherein the piezoelectric micromachined ultrasound transducer is a dual-electrode bimorph piezoelectric micromachined ultrasound transducer.

8. The system of claim 6, wherein the piezoelectric micromachined ultrasound transducer is operable at a resonant frequency ranging from about 2 MHz to about 10 MHz.

9. The system of claim 1, wherein the at least one ultrasonic transducer is in the form of an array of ultrasonic transducers.

10. The system of claim 9, wherein a protective material is positioned over the array of ultrasonic transducers, wherein the protective material comprises parylene, silicone, polydimethylsiloxane, or a combination thereof.

11. The system of claim 9, wherein the array of ultrasonic transducers includes a beam forming system for controlling beam steering angles.

12. The system of claim 9, wherein the array of ultrasonic transducers comprises at least four ultrasonic transducers.

13. The system of claim 1, the power component comprising a battery.

14. The system of claim 1, the ultrasonic sensor patch further comprising an attachment means configured for ensuring contact to the wearer.

15. A method for monitoring of blood pressure via a continuous, non-invasive, blood pressure monitoring system, the method comprising:
i) providing an ultrasonic sensor patch, the ultrasonic sensor patch comprising:
at least one ultrasonic transducer located externally on a body part of the wearer and in a vicinity of a vessel;
a power component;
at least one processor; and
a first memory including instructions capable of execution by the at least one processor;
ii) emitting an ultrasonic beam signal from an emitter associated with the at least one ultrasonic transducer;
iii) receiving a reflected ultrasonic beam signal by a detector associated with the at least one ultrasonic transducer;
iv) determining, via the at least one processor, the diameter of the vessel using the emitted ultrasonic beam signal and the reflected ultrasonic beam signal; and
v) determining, via the at least one processor, a cuffless blood pressure measurement for the wearer based on the diameter of the vessel.

16. The method of claim 15, further comprising:
transmitting the cuffless blood pressure measurement to a host computer via a network.

17. The method of claim 16, further comprising calibrating the continuous, non-invasive, blood pressure monitoring system by:
i) providing a calibration system, the calibration system comprising:
an inflatable cuff;
at least one controller;
a compressor in communication with the inflatable cuff;
a pressure transducer;
an ultrasonic Doppler sensor located externally on the body part of the wearer in the vicinity of the vessel and positioned proximal to the inflatable cuff; and
a second memory including instructions capable of execution by the at least one controller;
ii) inflating or deflating the cuff with the compressor;
iii) measuring the pressure levels to which the inflatable cuff is inflated or deflated in real time via the pressure transducer;
iv) determining a velocity of blood flow within the vessel located near the ultrasonic Doppler sensor;
v) adjusting the velocity of blood flow within the vessel to a predetermined set point by inflating or deflating the inflatable cuff via the compressor; and
vi) determining the pressure level to which the inflatable cuff is inflated or deflated to maintain the predetermined set point as measured via the pressure transducer, wherein the pressure level corresponds with a transmural blood pressure measurement for the wearer.

18. The method of claim 17, further comprising transmitting the transmural blood pressure measurement to the host computer via the network, wherein the transmural blood pressure is utilized to calibrate the continuous, non-invasive, blood pressure monitoring system to determine the cuffless blood pressure.

19. The method of claim 18, wherein the calibration system is disconnected from the continuous, non-invasive, blood pressure monitoring system once calibration has been completed.

20. The method of claim 17, wherein the ultrasonic transducer is a piezoelectric micromachined ultrasound transducer (PMUT), a capacitive micromachined ultrasonic transducer (CMUT), a lead zirconate titanate (PZT) transducer, or a combination thereof, wherein the ultrasonic transducer, the ultrasonic Doppler sensor, or both are operable at a resonant frequency ranging from about 2 MHz to about 10 MHz.
